(19) United States
(12) Patent Application Publication
(10) Pub. No.: US 2005/0239165 A1
(43) Pub. Date: Oct. 27, 2005

(54) Method for cultivation of the nitrile-hydratase-producing strain Rhodococcus rhodochrous M33

(75) Inventors: Josef Lorenz, Krefeld (DE); S.P. Woronin, Saratov (RU); S.W. Kosolin, Saratov (RU); I.N. Singirezev, Saratov (RU); V.G. Debabov, Moskow (RU); A.S. Yanenko, Moskow (RU)

Correspondence Address: OBLON, SPIVAK, MCCLELLAND, MAIER & NEUSTADT, P.C., 1940 DUKE STREET, ALEXANDRIA, VA, 22314, US

(73) Assignee: Stockhausen GmbH, Krefeld (DE)

(21) Appl. No.: 11/042,511

(22) Filed: Jan. 26, 2005

Related U.S. Application Data
(63) Continuation of application No. PCT/EP04/00361, filed on Apr. 6, 2004.

(51) Int. Cl. C12P 21/06; C12P 13/02; C12N 9/80; C12N 1/21

(52) U.S. Cl. 435/69.1; 435/228; 435/252.3

(57) ABSTRACT

The invention relates to a method for cultivation of the nitrile-hydratase-producing strain Rhodococcus rhodochrous M33, using a culture medium which is based on a 12 to 60 mM phosphate buffer, covers the demand of the cells for phosphorus and maintains the pH in the range of 5.5 to 9.0 during cultivation, and to which acetic acid is added in doses as the new source of carbon after consumption of the initially supplied quantity of glucose. The invention also relates to the culture medium used in the method.

### BACKGROUND OF THE INVENTION

1. Field of the Invention

The invention relates to a biotechnological method for cultivation of the nitrile-hydratase-producing strain Rhodococcus rhodochrous M33 and to a culture medium used in the method to increase the yield of biomass having high nitrile hydratase activity.

2. Description of the Background Art

The development of industrial biotechnological production methods for chemical compounds has led to great interest in microorganisms with special enzymes, which can be used for selected biocatalytic reactions. An example thereof is the biotechnological production method for synthesis of amides, which method is based on the ability of some microorganisms to synthesize nitrile hydratase, an enzyme that catalyzes the conversion of nitrites of organic acids to the corresponding amides. In this regard, bacterial strains that can transform the acrylonitrile to acrylamide are of particular interest.

The method for cultivation of the respective microorganisms plays an important role in production of the catalytically active biomass or biocatalyst. Usually two options are available for increasing the enzyme-activity yield per unit volume of culture broth (total activity in U/ml):

1. activating the enzyme synthesis of the (bacterial) cell, or in other words increasing the specific enzyme activity (U/mg dry biomass)

2. raising the yield of active cells (active biomass) per unit volume of the culture broth.

In British Patent 2087926 A and the patent literature cited therein, there are described among other methods the cultivation method for the Corynebacterium sp. N-774 strain (filing number FERM 4446 of the Fermentation Research Institute). The strain, which is accessible via a nitrile hydratase, is cultivated in a mineral medium containing a water-soluble iron compound in concentrations of at least 0.2 mg/l as well as 1 wt % of casein hydrolysate (casamino acids). The disadvantages of this method are the high costs of the casein hydrolysate and the low nitrile hydratase activity achieved:

specific activity—53.3 U/mg, dry biomass—5.66 mg/ml, total activity—301.7 U/ml.

It is known that nitriles and amides of organic acids, especially propionic and isobutyric acid, lead to induction of nitrile hydratase. In this regard, it can be inferred from U.S. Pat. No. 4,555,487 that the addition of such compounds, which are known as enzyme-inducing agents or inductors, to the culture medium of Pseudomonas chlororaphis strain B 23 (FERM BP-187) and Pseudomonas sp. strain PS 1 (FERM BP-188) makes it possible to obtain biomass (5.36 g/l of dry biomass) with a specific activity of 62.65 U/mg and a total activity of 335.8 U/ml.

The disadvantages of this method lie in the use of cost-intensive inductors and a low nitrile hydratase activity of the obtained cells.

The culture medium described in EP 0115781 for incubation of Pseudomonas chlororaphis strain B23 (FERM BP-187) and Pseudomonas sp. strain PS-1 (FERM BP-188) contains not only the inductors described, for example, in U.S. Pat. No. 4,555,487 but also one or more α-amino acids as agents for increasing the enzyme activity. For example, α-amino acids such as cystine, cysteine, alanine, valine, methionine, etc. are used at a concentration in the range of 0.1 to 10.0 g/l. Nevertheless, the specific activity of the biomass obtained in this way does not exceed the value of 105.7 U/mg, or the total activity does not exceed 428.1 U/ml.

A higher nitrile hydratase activity was achieved in incubation of Rhodococcus rhodochrous strain M33, which is described in German Patent 4480132. Therein it was shown that, during cultivation of the strain in synthetic medium containing glucose (concentration 5 g/l), it is possible to obtain cells with a specific activity of up to 200 U/mg as well as a total activity of up to 360 U/ml. A substantial advantage of the proposed method is the simplicity of the medium, which does not contain any expensive components. However, when the glucose concentration was increased to 20 g/l, the total activity did not exceed the value of 1368 U/ml.

A technically adequate method is the method described in European Patent 0362829 for cultivation of Rhodococcus rhodochrous strain J1 (FERM BP-1478). In the medium, which contains an inexpensive inductor—urea (concentration 15 g/l) and compounds of bivalent cobalt—the proposed cultivation method makes it possible to obtain cells with a specific activity of 578 U/mg. To increase the cell yield (biomass yield), peptone and yeast extract are additionally admixed with the medium. However, the values of total activity do not exceed 2480 U/ml, because the biomass yield is only 4.3 g/l. In “upscaling” of the cultivation process under industrial conditions, the cell yield was increased to 28 g/l; at the same time, the specific nitrile hydratase activity decreased to 76 U/mg and the total activity to 2100 U/ml (Yamada H., Kobayashi M., “Nitrile hydratase and application to industrial production of acrylamide”, Biosci. Biotech. Biochem., 60 (9), 1391-1400, 1996. Applications of nitrile hydratase to the industrial production of acrylamide as well as other amides, including nicotinamide, and the action of nitrile hydrase on aliphatic and aromatic nitriles, are described by Yamada et al., id, see e.g., pages 1398-1399, which is hereby incorporated by reference.

A further method for cultivation of Rhodococcus rhodochrous strain M33 is described in the literature (Kim B-Y., Kim J-C., Lee H-H., Hyun H-H., “Fed-batch Fermentation for Production of Nitrile Hydratase by Rhodococcus rhodochrous M33”, Biotechnol. Bioprocess Eng., 6: 11-17, 2001). This method comprises a glucose-limited, fed-batch fermentation in a 5-liter fermenter with a purely synthetic culture medium consisting of KH₂PO₄, K₂HPO₄, FeSO₄×7 H₂O, EDTA-2Na, MgSO₄×7 H₂O, NaCl, CoCl₂×6 H₂O, urea and glucose. By constant addition of doses of a 40% glucose solution and three periodic additions of CoCl₂×6 H₂O (0.01 g/l) to the glucose solution, a cell yield of 24 g/l and a specific nitrile hydratase activity of 120 U/mg were achieved. Nevertheless, the total activity after a relatively long fermentation time of approximately 115 hours did not exceed the value of 2880 U/ml.

Thus a need still exists for biotechnological methods for cultivation of nitrile-hydratase-producing strains that make it possible, under industrial conditions, to increase the yield of biomass having high nitrile hydratase activity.

One object of the present invention was to discover such a method.

### SUMMARY OF THE INVENTION

Surprisingly, it has been found that a method for cultivation of the nitrile-hydratase-producing Rhodococcus rhodochrous, for example, strain M33, which is deposited under deposition number DSM 14230 with the DSMZ, Deutsche Sammlung von Mikroorganismen und Zellkulturen GmbH, Marschroder Weg 1b, D-38124 Braunschweig, Germany, achieves the inventive object when there is used for cultivation a culture medium which is based on a 12 to 60 mM phosphate buffer, covers the demand of the cells for phosphorus and maintains the pH in the range of 5.5 to 9.0 during cultivation, and to which acetic acid is added in doses as the new source of carbon after consumption of the initially supplied quantity of glucose.

### DETAILED DESCRIPTION OF THE PREFERRED EMBODIMENTS

According to the invention, there is used a culture medium which covers the demand of the cells for phosphorus and makes it possible to maintain the pH in the range of 5.5 to 9.0 during cultivation. Preferably such a culture medium contains corn extract as an additional growth factor.

In the inventive method, it is particularly preferable to use a corn extract as an additional growth factor in the culture medium, by adding it in the form, for example, of waste solution from cornstarch production or corn steep liquor in a concentration such as 1 to 10 g/l. All waste solutions from cornstarch production that contain the constituents of the corn grains dissolved in concentrated form by means of a standard steeping process are usable as corn extracts. According to the invention, there are preferably used inventive waste solutions from cornstarch production sold under the name Cerestar 15840 by Cerestar Deutschland GmbH, Krefeld.

The culture medium (abbreviated as SI hereinafter) that can be used in the inventive method preferably has the following composition (in g/l):

a.) 7.9 g/l of Na₂HPO₄×12 H₂O

b.) 1.8 g/l of KH₂PO₄

c.) 0.02 to 0.04 g/l of CoCl₂×6 H₂O

d.) 1.0 g/l of MgSO₄×7 H₂O

e.) 0 to 10 g/l of corn extract

f.) 20 to 90 g/l of glucose

g.) 4 to 20 g/l of urea

In particular, there will be used such a culture medium to which the corn extract has been added in a concentration of 1 to 10 g/l, preferably 2 to 8 g/l and particularly preferably 2 to 6 g/l. Quite particularly preferably the corn extract can be used in a concentration of 4 g/l in the culture medium.

The basis of this culture medium is a 12 to 60 mM phosphate buffer, which covers the demand of the cells for phosphorus and ensures that the pH can be maintained in the range of 5.5 to 9.0 during cultivation, glucose and acetic acid being used as sources of carbon and acetic acid being used as a new additional source of carbon besides glucose.

Particularly preferably, a 35.3 mM phosphate buffer is used for this purpose in the inventive method.

Advantageously the culture medium contains 20 to 90 g/l, preferably 20 to 60 g/l and particularly preferably 20 to 40 g/l of glucose as the source of carbon, especially for cultivation in shaking flasks. Besides glucose, acetic acid can be used as a source of carbon, the sources of carbon preferably being added in the form of a two-phase process, especially for cultivation in fermenters—in the laboratory—up to large industrial scale.

The first phase of this process is characterized by initiated, intensified cell growth of Rhodococcus rhodochrous M33 on the basis of the first source of carbon initially supplied in the medium, preferably 20 g/l of glucose. After consumption of the glucose, the fermentation is continued in the second phase in what is known as a fed-batch process, in which acetic acid is used as the source of carbon. The less expensive acetic acid can replace the glucose that is usable for cultivation, and it ensures a high specific nitrile hydratase activity and a high total activity.

Advantageously the acetic acid can also be added in the form of its water-soluble salts, sodium acetate being particularly preferred as a water-soluble acetate salt. In one embodiment of the invention, the water-soluble acetate salt to be used according to the invention is added in doses after consumption of the glucose, in which case the added concentration of sodium acetate, which is to be used preferably, can be 1 to 4 g/l. An added concentration of 3 g/l of sodium acetate is particularly preferred for this purpose. In a further embodiment of the inventive method, the water-soluble salt of acetic acid, preferably sodium acetate, is initially supplied first. After the pH has risen to 7.5 to 8.5, acetic acid is then added in doses as a 20 to 50%, preferably 30% aqueous solution. Advantageously, the aqueous acetic acid solution is added in doses by pH-stat feeding at a pH of 7.5 to 8.5, especially at a pH of 7.9.

In another way in which the inventive method can be performed, the components to be added, preferably CoCl₂×6 H₂O and urea, can be added in doses at certain times in suitable concentrations during fermentation, the cobalt acting as a cofactor and the urea as an inductor.

In particular, 4 to 20 g/l of urea and 0.02 to 0.04 g/l of CoCl₂×6 H₂O are added in doses to the culture medium for induction of the nitrile hydratase. Preferably the urea addition takes place by initially supplying some urea as an inductor in the medium, especially in a feed concentration of 4 g/l, and then adding more in doses, preferably in a concentration of 6 g/l, after consumption of the initially supplied glucose.

According to the invention, the addition of the CoCl₂×6 H₂O cofactor can take place by admixing the CoCl₂×6 H₂O, preferably in a feed concentration of 0.01 g/l, with the medium at the beginning of the exponential growth phase, which is based on glucose as the source of carbon, and then, after consumption of the glucose, adding more in doses, preferably in a feed concentration of 0.03 g/l.

It was surprisingly found that shortening of the fermentation time can be achieved if the inventive method is carried out by adding CoCl₂×6 H₂O in doses, preferably of 0.01 g/l, at the beginning of the exponential growth phase.

Strain cultivation by the inventive method takes place in baffle flasks (shaking cultures) and in laboratory and industrial fermenters in the temperature range of 25 to 30° C. for a cultivation time of 48 to 170 hours. In a preferred embodiment of the inventive method, the oxygen supply during cultivation, especially in industrial fermenters, is designed such that the relative O₂ partial pressure is greater than 30%, preferably ≦50%.

The advantages of the inventive method for cultivation of the nitrile-hydratase-producing strain Rhodococcus rhodochrous M33 are that, by using the inventive culture medium, especially with corn extract as an additional growth factor, not only can large quantities of nitrile-hydratase-containing biomass (yield up to 39.5 g/l of dry biomass) be produced, but so also can a biomass having high specific nitrile hydratase activity (up to 315 U/mg), while a nitrile hydratase total activity of 7169 U/ml can be achieved, as can be inferred from the examples hereinafter.

In contrast to the known method for cultivation of nitrile-hydratase-producing strains, the present method according to the invention is characterized by the use of a culture medium based on a 12 to 60 mM phosphate buffer, which covers the demand of the cells for phosphorus and maintains the pH in the range of 5.5 to 9.0 during cultivation, and acetic acid added in doses as the new source of carbon after consumption of the initially supplied quantity of glucose. Moreover, corn extracts in the form of waste solution from cornstarch production or corn steep liquor can be used as an additional growth factor. Furthermore, it is advantageous that the glucose that can be used for cultivation in the culture medium can be replaced by the less expensive acetic acid, and so a high specific nitrile hydratase activity and high total activity of the inventive method is assured.

Standard Test for Measurement of the Nitrile Hydratase Activity

1 ml of 2% acrylonitrile solution in 10 mM phosphate buffer (pH 7.5) is mixed with 1 ml of M33 cell suspension obtained by previous centrifugation of the culture broth, washing and then resuspension of the cells in 10 mM phosphate buffer (pH 7.5). The suspension contains 0.08 to 0.16 mg of cells (dry mass). The reaction runs for 5 minutes at 20° C., after which the reaction is stopped by addition of 20 μl of concentrated HCl. The concentration of the acrylamide formed is determined by gas chromatography or photometry.

The nitrile hydratase activity is reported in the following units.

$$specific activity - in μmol acrylamide / min \times mg dry biomass = [ U \text{/} mg ]$$

$$total activity - in μmol acrylamide / min \times ml culture broth = [ U \text{/} ml ]$$

The invention is illustrated by means of the following examples:

### EXAMPLE 1

Baffle flasks (250 ml) with 50 ml of culture medium “SI” (composition in g/l: Na₂HPO₄×12 H₂O—7.9; KH₂PO₄—1.8; CoCl₂×6 H₂O—0.02; MgSO₄×7 H₂O—1.0; corn extract—2.0; pH 7.2±0.2) containing glucose (50 g/l) and urea (4 to 20 g/l) were inoculated with 1 ml of Rhodococcus rhodochrous M33 inoculum culture. The inoculum was incubated within 24 hours in the same medium. Cultivation took place in a shaking incubator at 180 rpm (circular) and 30° C. within 96 hours. The yield of biomass and the nitrile hydratase activity of the cells were determined. The results are presented in Table 1.

TABLE 1

| | |
|---|---|
| Dry | |
| biomass | Nitrile hydratase activity |

| Urea | yield | Specific activity | Total activity |
|---|---|---|---|
| concentration [g/l] | [g/l] | [U/mg] | [U/ml] |
| 4 | 22.3 | 62 | 1383 |
| 8 | 24.2 | 126 | 3040 |
| 12 | 24.4 | 150 | 3660 |
| 16 | 25.1 | 177 | 4453 |
| 20 | 23.0 | 173 | 3970 |

### EXAMPLE 2

Baffle flasks (250 ml) with 50 ml of culture medium “SI” (composition in g/l: Na₂HPO₄×12 H₂O—7.9; KH₂PO₄—1.8; CoCl₂×6 H₂O—0.02; MgSO₄×7 H₂O—1.0; urea—16.0; pH 7.2±0.2) containing glucose (50 g/l) and corn extract (0 to 10 g/l) were inoculated with 1 ml of Rhodococcus rhodochrous M33 inoculum culture. The inoculum was incubated within 24 hours in the same medium. Cultivation took place in a shaking incubator at 180 rpm (circular) and 30° C. within 96 hours. The yield of biomass and the nitrile hydratase activity of the cells were determined and the results are presented in Table 2.

TABLE 2

| | | |
|---|---|---|
| | Dry | |
| Corn extract | biomass | Nitrile hydratase activity |

| concentration | yield | Specific activity | Total activity |
|---|---|---|---|
| [g/l] | [g/l] | [U/mg] | [U/ml] |
| 0* | 23.3 | 126 | 2940 |
| 1 | 23.5 | 140 | 3299 |
| 2 | 24.4 | 150 | 3660 |
| 4 | 24.9 | 172 | 4290 |
| 6 | 24.6 | 163 | 4012 |
| 8 | 24.4 | 156 | 3802 |
| 10 | 25.3 | 141 | 3562 |

0.01 g/l of FeSO₄×7 H₂O was added in doses to this starting mixture.

From the foregoing data, it is evident that the addition of corn extract leads to an increase of specific and total nitrile hydratase activity. The optimal concentration of the corn extract is 4 g/l.

### EXAMPLE 3

Baffle flasks (250 ml) with 50 ml of culture medium “SI” (composition in g/l: Na₂HPO₄×12 H₂O—7.9; KH₂PO₄—1.8; CoCl₂×6 H₂O—0.02; MgSO₄×7 H₂O—1.0; urea—16.0; corn extract—4.0; pH 7.2±0.2) contain glucose in concentrations of 20 to 90 g/l. Inoculation and incubation of the flasks took place as in Example 1. The measured biomass yield and the nitrile hydratase activity of the cells are summarized in Table 3.

TABLE 3

| | Dry | | | |
|---|---|---|---|---|
| Glucose | biomass | Incubation | Nitrile hydratase activity | |
| concentration | yield | time | Specific activity | Total activity |
| [g/l] | [g/l] | [hours] | [U/mg] | [U/ml] |
| 20 | 10.5 | 72 | 306 | 3212 |
| 30 | 15.9 | 72 | 255 | 4051 |
| 40 | 19.2 | 96 | 223 | 4282 |
| 50 | 23.5 | 96 | 216 | 5076 |
| 60 | 27.4 | 120 | 204 | 5595 |
| 70 | 32.7 | 144 | 198 | 6465 |
| 80 | 36.4 | 166 | 191 | 6956 |
| 90 | 39.5 | 166 | 182 | 7169 |

As follows from Table 3, the cell yield and the total nitrile hydratase activity increase in proportion to the increase in glucose concentration in the culture medium. This results from the fact that all components of the culture medium are present in non-limiting concentrations.

### EXAMPLE 4

A 3-liter laboratory fermenter with 1.5 liters of medium “SI” (composition in g/l: Na₂HPO₄×12 H₂O—7.9; KH₂PO₄—1.8; CoCl₂×6 H₂O—0.01; MgSO₄×7 H₂O—1.0; urea—16.0; corn extract—4.0; pH 7.2±0.2) containing 5 g/l of glucose was inoculated with 100 ml of Rhodococcus rhodochrous M33 inoculum culture. The inoculum was incubated within 24 hours in a medium of the same composition.

Cultivation conditions:

| | |
|---|---|
| temperature | 30° C. |
| stirrer speed | 560 rpm |
| aeration rate | 1.5 vvm (volume/volume/minute) |

After a fermentation time of 18 hours, 1.5 g/l of glucose was added in doses every 2 hours up to a final concentration of 40 g/l. As a result, it was found that 16.4 g/l of dry biomass with a specific activity of 215 U/mg had been produced. The total activity was 3526 U/ml.

### EXAMPLE 5

A 3-liter laboratory fermenter with 1.5 liters of medium “SI” (composition in g/l: Na₂HPO₄×12 H₂O—7.9; KH₂PO₄—1.8; CoCl₂×6 H₂O—0.01; MgSO₄×7 H₂O—1.0; urea—4.0; corn extract—4.0; pH 7.2±0.2) containing 20 g/l of glucose was inoculated as in Example 4. The cultivation conditions were also as in Example 4. After a fermentation time of 24 to 36 hours and consumption of the entire quantity of glucose, 6 g/l of urea, 3 g/l of sodium acetate and 0.03 g/l of CoCl₂×6 H₂O were added in doses. After the pH of the medium had risen, 30% acetic acid was added in doses (fed batch). Under these conditions, it was found that 19.6 g/l of dry biomass with a specific activity of 315 U/mg had been produced within 72 hours. The total activity was 6174 U/ml.

### EXAMPLE 6

A 15-liter laboratory fermenter with 7.0 liters of medium “SI” (composition in g/l: Na₂HPO₄×12 H₂O—7.9; KH₂PO₄—1.8; MgSO₄×7 H₂O—1.0; urea—4.0; corn extract—4.0; pH 7.2±0.2) containing 20 g/l of glucose was inoculated with 4% (v/v) of Rhodococcus rhodochrous M33 inoculum culture. The inoculum was incubated within 24 hours as a shaking culture in a medium of the same composition.

Cultivation conditions:

| | |
|---|---|
| temperature | 29 to 30° C. |
| aeration rate | 0.5 vvm |
| overlay pressure | 0.3 bar |
| relative O₂ partial pressure | ≧50% (controlled via stirrer speed) |
| stirrer speed | 300 to 600 rpm |

After a fermentation time of 12 hours (beginning of the exponential growth phase), 0.01 g/l of CoCl₂×6 H₂O was added in doses. After a fermentation time of 21 to 23 hours and consumption of the entire quantity of glucose, 6 g/l of urea, 3 g/l of Na acetate and 0.03 g/l of CoCl₂×6 H₂O were added in doses. After the pH of the culture broth had risen to 7.9, the new source of carbon in the form of acetic acid (30%) was added in doses with regulation of the pH to 7.9 (pH-stat feeding). Under these conditions, it was found that 19.9 g/l of dry biomass with a specific activity of 294 U/mg had been produced within only 66.5 hours. The total activity was 5851 U/ml.

By the fact that CoCl₂×6 H₂O was admixed with the medium for the first time at the beginning of the exponential growth phase, it was possible to shorten the fermentation time compared with Example 5.

### EXAMPLE 7

An industrial 15-m³ production fermenter with 10 m³ of culture medium “SI” (composition in g/l: H₃PO₄—3.46; KOH—0.74; NaOH—for adjustment of the pH to 6.9; CoCl₂×6 H₂O—0.01; MgSO₄×7 H₂O—1.0; urea—4.0; corn extract—4.0; pH 7.2±0.2) containing 20 g/l of glucose was inoculated with 5% (v/v) of Rhodococcus rhodochrous M33 inoculum culture. Incubation of the inoculum had been completed previously within 24 hours, in a prefermenter, also with medium “SI”.

Cultivation conditions:

| | |
|---|---|
| temperature | 28 to 30° C. |
| stirrer speed | 160 to 165 rpm |
| aeration rate | 0.5 vvm |

After 24 to 36 hours and consumption of the entire quantity of glucose, 6 g/l of urea, 3 g/l of sodium acetate and 0.03 g/l of CoCl₂×6 H₂O were added in doses to the medium. Feeding of the acetic acid took place by analogy with Example 6. After a fermentation time of 72 hours, it was found that 18.8 g/l of dry biomass with a specific activity of 290 U/mg had been produced. The total activity was 5452 U/ml.

Modifications and Other Embodiments

Various modifications and variations of the described cultivation methods and bacterial strains, as well as the concept of the invention will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the microbiological, bacteriological, fermentation, biological, chemical or related fields are intended to be within the scope of the following claims.

Incorporation by Reference

The priority document PCT/EP2004/003611 and German patent application DE 103 15 376 are hereby incorporated by reference in their entireties.

1. A method for cultivating a nitrile-hydratase-producing Rhodococcus rhodochrous and/or for making nitrile-hydratase comprising:
cultivating a nitrile-hydratase-producing strain of Rhodococcus rhodochrous in a medium containing a phosphate buffer and a first carbon source, and
adding acetic acid and/or a salt(s) of acetic acid to the culture medium after depletion or consumption of the first carbon source and, optionally,
isolating or recovering nitrile-hydratase.

2. The method of claim 1, wherein said Rhodococcus rhodochrous strain is M33.

3. The method of claim 1, wherein said medium contains 12 to 60 mM phosphate buffer.

4. The method of claim 1, wherein said first carbon source is glucose.

5. The method of claim 1, wherein said first carbon source comprises glucose and acetic acid.

6. The method of claim 1, wherein said medium contains 20-90 g/l of glucose.

7. The method of claim 1, wherein said medium further contains at least one corn extract.

8. The method of claim 1, wherein said medium contains a waste solution form cornstarch production and/or corn steep liquor.

9. The method of claim 1, wherein said medium contains 1 to 10 g/l of at least one corn extract.

10. The method of claim 1, wherein acetic acid is added to the culture medium after depletion or consumption of the first carbon source.

11. The method of claim 1, wherein at least one water soluble salt of acetic acid is added to the culture medium after the depletion or consumption of the first carbon source.

12. The method of claim 1, wherein sodium acetate is added to the culture medium after the depletion or consumption of the first carbon source.

13. The method of claim 1, wherein the acetic acid or salt of acetic acid is added after the culture medium has risen to pH 7.5 to 8.5.

14. The method of claim 1, wherein the acetic acid and/or salt of acetic acid is added to a concentration of 1 to 4 g/l after depletion or consumption of the first carbon source.

15. The method of claim 1, wherein the acetic acid and/or acetic acid salt is added by pH-stat feeding at a pH of 7.5 to 8.5.

16. The method of claim 1, further comprising adding urea to the culture medium before and/or during cultivation.

17. The method of claim 1, further comprising adding CoCl₂×6 H₂O to the culture medium during cultivation.

18. The method of claim 1, wherein CoCl₂×6 H₂O is admixed with the medium at the beginning of the exponential growth phase and added after depletion or consumption of the first carbon source.

19. The method of claim 1, which is conducted in a fermented in which the relative oxygen partial pressure is greater than 30-50%.

20. The method of claim 1, wherein nitrile-hydratase is part of the culture biomass.

21. The method of claim 1, wherein the nitrile hydratase is isolated or purified from components of the culture biomass.

22. A culture medium comprising:
at least one phosphate buffer,
0.02 to 0.04 g/l of CoCl₂×6 H₂O,
1.0 g/l of MgSO₄×7 H₂O,
0 to 10 g/l of corn extract,
20 to 90 g/l of glucose,
4 to 20 g/l of urea, and optionally,
1 to 10 g/l corn extract and/or optionally acetic acid.

23. The medium of claim 22, wherein the at least one phosphate buffer comprises 7.9 g/l of Na₂HPO₄×12 H₂O and 1.8 g/l of KH₂PO₄.

24. A method for converting a nitrile of an organic acid to the corresponding amide, comprising contacting a nitrile of an organic acid with the Rhodococcus rhodochrous or with a nitrile hydratase produced by the method of claim 1 for a time and under conditions suitable for the formation of the corresponding amide.

25. The method of claim 24, wherein the nitrile of an organic acid is acrylonitrile and the corresponding amide is acrylamide.
